(19) United States
(12) Patent Application Publication
(10) Pub. No.: US 2014/0271606 A1
(43) Pub. Date: Sep. 18, 2014

(54) METHODS OF USING ADENOSINE RECEPTOR ANTAGONISTS FOR TREATING BLEEDING DISORDERS

(71) Applicant: Bayer HealthCare LLC, Whippany, NJ (US)

(72) Inventor: Derek Sim, Burlingame, CA (US)

(21) Appl. No.: 14/180,113

(22) Filed: Feb. 13, 2014

Related U.S. Application Data
(60) Provisional application No. 61/786,692, filed on Mar. 15, 2013.

(51) Int. Cl. A61K 31/53; A61K 38/48; A61K 38/37

(52) U.S. Cl. CPC A61K 31/53; A61K 38/37; A61K 38/4846
USPC 424/94.64; 514/246; 514/14.1

(57) ABSTRACT

Methods of treating bleeding disorders, such as bleeding diseases such as hemophilia, by administering adenosine 2a receptor and/or adenosine 2b receptor antagonists to subjects in need thereof are disclosed. In some embodiments, the methods further include administration of the antagonist with one or more of Factor VIII, Factor IX and Factor XI to treat the bleeding disorder.

### BACKGROUND OF THE DISCLOSURE

The present disclosure relates generally to methods for restoring hemostasis in bleeding disorders. More particularly, the present disclosure relates to a method for treating bleeding disorders, and in particular, hemophilia, by administering to a subject in need thereof an adenosine receptor antagonist, and in particular, a platelet adenosine 2a receptor antagonist and/or a platelet adenosine 2b receptor antagonist. In some embodiments, the methods further include administration of the adenosine receptor antagonist with one or more of Factor VIII, Factor IX, and Factor XI.

Platelets (or thrombocytes) are small, irregularly shaped cell fragments derived from megakaryocytes that circulate in the blood of mammals and participate in hemostasis. In normal hemostasis, endothelial cells that line the inner surface of blood vessels prevent platelet activation by producing nitric oxide, endothelial-ADPase and PGI2. When the endothelial layer is injured, collagen, von Willebrand factor (vWF) and tissue factor from the subendothelium is exposed to the bloodstream. Upon contact with collagen or vWF, platelets are activated. After the initial adhesion of platelets to extracellular matrix at sites of vascular injury, autocrine and paracrine factors, including ADP, thrombin, epinephrine and thromboxane A2, amplify and sustain the initial platelet response and recruit circulating platelets to form a hemostatic clot. Thus, activation of platelets is a critical component in the formation of a blood clot (thrombosis) to prevent blood loss. Agonists such as ADP, thrombin and epinephrine directly activate platelet Gi receptors to result in a decrease in cAMP. Adenosine, which acts through abundant adenosine 2a and 2b receptors on platelet surfaces, increases intracellular cAMP levels to inhibit platelet activation.

Conventionally, treatment efforts for abnormal hemostasis, such as found in subjects with bleeding disorders (e.g., hemophilia, von Willebrand Disease, etc.) or suffering severe trauma, have focused on the activation of the coagulation cascade system. The coagulation cascade system, shown in FIG. 1, involves two initial pathways, the extrinsic pathway and the intrinsic pathway. The extrinsic pathway involves tissue factor and Factor VII complex to activate Factor X, while the intrinsic pathway involves high-molecular weight kininogen, prekallikrein, and Factors XII, XI, IX and VIII to activate Factor X. Both the extrinsic and intrinsic pathways lead to a final common pathway in which Factor X mediates the generation of thrombin from prothrombin, with the ultimate production of fibrin from fibrinogen.

Platelets and endothelial cells also play important roles in hemostasis, and therefore, in bleeding disorders, it is now believed that normal hemostasis could potentially further be restored by modulating platelet and endothelial cell activities. More particularly, it is now believed that platelet activation and coagulation can be increased at the surface of platelets, thereby generating a pro-hemostatic effect.

While treatments of bleeding disorders through the use of modulating/enhancing the coagulation cascade system have met with success, there exists a need to develop alternative mechanisms to restore hemostasis. Particularly, methods for activating platelets and increasing platelet surface coagulation at the platelet surface itself could prove advantageous in treating bleeding disorders. These methods alone, or in combination with conventional pro-coagulation cascade treatments, may provide for more efficient and effective generation of pro-hemostatic effects in subjects suffering from bleeding disorders and in other bleeding situations.

### SUMMARY OF THE DISCLOSURE

The present disclosure is generally directed to treating bleeding disorders. More particularly, the present disclosure relates to a method for treating bleeding disorders, such as bleeding diseases including hemophilia, by administering an adenosine receptor antagonist to subjects in need thereof. It has been unexpectedly discovered that by targeting adenosine receptors (e.g., adenosine 2a and 2b receptors), pro-hemostatic conditions can be effectively restored without solely targeting the coagulation cascade system Inhibition of platelet adenosine receptor binding leads to less intracellular cAMP levels, thereby increasing platelet activation and enhancing the level of coagulation on the platelet surface. In turn, bleeding time and total blood loss in the subject is reduced. In some embodiments, the methods can further include co-administering Factor VIII, Factor IX, and/or Factor XI with the adenosine receptor antagonists to treat the bleeding disorder.

In one aspect, the present disclosure is directed to a method for treating a bleeding disorder in a subject in need thereof. The method comprises administering a therapeutically effective amount of an adenosine receptor antagonist.

In another aspect, the present disclosure is directed to a method for reducing bleeding time in a subject in need thereof. The method comprises administering a therapeutically effective amount of an adenosine receptor antagonist.

In yet another aspect, the present disclosure is directed to a method for treating hemophilia in a subject in need thereof. The method comprises administering a therapeutically effective amount of an adenosine receptor antagonist.

In yet another aspect, the present disclosure is directed to a method for enhancing platelet aggregation in response to adenosine diphosphate (ADP) in a subject in need thereof. The method comprises administering a therapeutically effective amount of an adenosine receptor antagonist.

In yet another aspect, the present disclosure is directed to a method for increasing platelet activation in a subject in need thereof. The method comprises administering a therapeutically effective amount of an adenosine receptor antagonist.

In yet another aspect, the present disclosure is directed to a method for increasing platelet surface coagulation in a subject in need thereof. The method comprises administering a therapeutically effective amount of an adenosine receptor antagonist.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a schematic of the conventional coagulation cascade system.

FIG. 2 is a graph depicting median blood loss of Hemophilia A mice after intraperitoneal (IP) administration of 100 mg/kg ZM241385 or excipient (100% DMSO) as discussed in Example 1.

FIG. 3 is a graph depicting median blood loss of Hemophilia A mice after intraperitoneal (IP) administration of 300 mg/kg ZM241385 or excipient (40/10/50 v/v/v PEG400/ethanol/water) as discussed in Example 1.

FIG. 4 is a graph depicting median blood loss of Hemophilia B mice after intravenous (IV) administration of 100 mg/kg ZM241385 or excipient (60/40 v/v PEG400/water) as discussed in Example 1.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

In accordance with the present disclosure, methods have been discovered that surprisingly allow for the treatment of bleeding disorders by targeting adenosine receptors on platelet surfaces. Particularly, antagonists that specifically bind to adenosine receptors are administered, inhibiting adenosine receptor activation. This inhibition causes less adenosine to bind to adenosine receptors, which reduces the intracellular production of cAMP and allows for increased levels of platelet activation and platelet surface coagulation to be achieved. The methods provide for reduced bleeding time and more efficient and effective generation of normal hemostasis in subjects in need thereof.

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In accordance with the present disclosure, methods have been discovered that restore normal hemostasis for bleeding disorders by inhibiting targets unrelated to the coagulation cascade. In one aspect, the present disclosure is directed to a method for treating a bleeding disorder in a subject in need thereof. The method includes administering a therapeutically effective amount of an adenosine receptor antagonist.

As used herein, “adenosine receptor antagonist” refers to a high affinity antagonist that is selective for the adenosine receptor; that is, that specifically binds to an adenosine receptor. In one embodiment, the antagonist is selective for the adenosine 2a receptor. In another embodiment, the antagonist is selective for the adenosine 2b receptor. In yet another embodiment, the antagonist is selective for both the adenosine 2a receptor and the adenosine 2b receptor.

As used herein, “bleeding disorder” refers to a disease or condition that impairs normal homeostasis. The bleeding disorder can be, for example, Hemophilia A, Hemophilia B, Factor VIII deficiency, Factor XI deficiency, von Willebrand Disease, Glanzmann's Thrombasthenia, Bernard Soulier Syndrome, idiopathic thrombocytopenic purpura, trauma (including intracerebral hemorrhage and traumatic brain injury) and the like.

As used herein, “hemophilia” refers to a group of bleeding disorders associated with increased blood clot formation time as compared to blood clot formation time in healthy individuals without hemophilia. “Hemophilia” refers to both Hemophilia A, which is a disorder that leads to the production of defective Factor VIII, and Hemophilia B, which is a disorder that leads to the production of defective Factor IX.

As used herein, “trauma” refers to an injury resulting in sudden and severe blood loss. Exemplary traumas are known in the art and include, but are not limited to, intracerebral hemorrhage, traumatic brain injury, blunt trauma, penetrating trauma, and the like.

As used herein, “reducing bleeding time”, “reduced bleeding time” and “reduction in bleeding time” refer to the shortening of the time period required for clot formation in a subject such to arrest bleeding, and thus provide a reduced bleeding volume, by administration of an adenosine receptor antagonist in comparison to a subject who does not receive the antagonist.

As used herein, “pro-hemostatic condition” refers to the restoration of clot formation such to arrest bleeding. Further, “generation of a pro-hemostatic condition” refers to the restoration/generation of normal hemostasis in a subject by administration of an adenosine receptor antagonist in comparison to a subject who does not receive the adenosine receptor antagonist.

As used herein, “normal hemostasis” refers to the hemostasis process of a healthy subject not suffering from a bleeding disorder as defined herein.

As used herein, “susceptible” and “at risk” refer to having little resistance to a certain disease, disorder or condition, including being genetically predisposed, having a family history of, and/or having symptoms of the disease, disorder or condition.

As used herein, in the most general form, “specific binding”, “binds specifically to”, “specific to/for” or “specifically recognizes” refer to the ability of the antagonist to discriminate between the adenosine receptor and an unrelated receptor, as determined in accordance with methods known in the art, such as, for example, selectivity profiling using cell based assays (e.g., Ricerca cell-based screen).

As used herein, “binding affinity” refers to the strength of the sum total of non-covalent interactions between a single binding site of a molecule and its binding partner. Unless indicated otherwise, as used herein, “binding affinity” refers to intrinsic binding affinity which reflects a 1:1 interaction between members of a binding pair (e.g., an antagonist and receptor). The dissociation constant “K_{D}” is commonly used to describe the affinity between a molecule (such as an antagonist) and its binding partner (such as a receptor), i.e., how tightly a ligand binds to a particular protein. Ligand-protein affinities are influenced by non-covalent intermolecular interactions between the two molecules. Affinity can be measured by common methods known in the art, including, for example, surface plasmon resonance or isothermal titration calorimetry.

All combinations of method or process steps as used herein can be performed in any order, unless otherwise specified or clearly implied to the contrary by the context in which the referenced combination is made.

### Methods of Administering Adenosine Receptor Antagonists

The methods of the present disclosure generally include the administration of one or more adenosine receptor antagonists to a subject in need thereof to promote/restore/maintain the hemostasis process during bleeding. Particularly, the methods of the present disclosure can prevent/control/reduce/treat bleeding disorders in subjects in need thereof; that is, by promoting/restoring/maintaining clot formation, the methods can prevent/reduce/control bleeding time, thereby preventing/controlling/reducing/treating bleeding disorders. As a further result, the methods of the present disclosure can control/reduce/minimize bleeding time, and also, the amount of total blood loss (control/reduce/minimize blood volume) when bleeding occurs.

Adenosine normally binds to adenosine receptors on platelets to stimulate adenylyl cyclase. Adenylyl cyclase in platelets increases intracellular cAMP, which is a potent inhibitor of platelet activation. Accordingly, by binding to adenosine receptors and decreasing the activity of adenylyl cyclase, the methods of the present disclosure can additionally reduce/prevent/control cAMP generation as compared to subjects that are not administered the adenosine receptor antagonist. Further, by reducing cAMP generation, the methods of the present disclosure can increase/enhance/promote levels of platelet aggregation, increase/enhance/promote levels of platelet activation and increase/enhance/promote levels of coagulation on platelet surface as compared to subjects that are not administered the adenosine receptor antagonist.

The adenosine receptor antagonist described below in detail and used in the methods of the present disclosure can be administered to a subset of subjects in need of promoting/restoring/maintaining the hemostasis process. Some subjects that are in specific need of restored/maintained hemostasis may include subjects who are susceptible to, or at elevated risk of, experiencing bleeding situations, including subjects susceptible to, or at elevated risk of, bleeding disorders such as Hemophilia A, Hemophilia B, Factor VIII deficiency, Factor XI deficiency, von Willebrand Disease, Glanzmann's Thrombasthenia, Bernard Soulier Syndrome, idiopathic thrombocytopenic purpura, intracerebral hemorrhage, trauma, traumatic brain injury, and the like. In one particular embodiment, the methods can be administered to a subject who has, or is susceptible to, or at elevated risk of, hemophilia. Subjects may be susceptible to, or at elevated risk of, experiencing bleeding situations due to family history, age, environment, and/or lifestyle. Additionally, the methods can be administered to a subject to modulate vasodilation, modulate dopaminergic activity in central nervous system, modulate neuron excitation, or modulate bronchospasms. Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

The adenosine receptor antagonist can be administered alone in a suitable pharmaceutical formulation (i.e., no other active compound) or as a component of a suitable pharmaceutical formulation comprising the antagonist in combination with another active compound such as Factor VIII, Factor IX and/or Factor XI, which are described more fully below. Additionally, the adenosine receptor antagonist, alone or in combination with another active compound such as Factor VIII, Factor IX and/or Factor XI, may be used in the manufacture of one or more medicaments. The pharmaceutical formulations may include one or more pharmaceutically acceptable carriers as are known in the art. As used herein, the phrase “pharmaceutically acceptable” refers to those ligands, materials, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier”, as used herein, refers to a pharmaceutically acceptable material, formulation or vehicle, such as a liquid or solid filler, diluent, excipient, solvent or encapsulating material, involved in carrying or transporting the active compound from one organ or portion of the body, to another organ or portion of the body. Each carrier must be “acceptable” in the sense of being compatible with the other components of the formulation and not injurious to the subject. Lyophilized formulations, which may be reconstituted and administered, are also within the scope of the present disclosure.

Pharmaceutically acceptable carriers may be, for example, excipients, vehicles, diluents, and combinations thereof. For example, where the formulations are to be administered orally, they may be formulated as tablets, capsules, granules, powders, or syrups; or for parenteral administration, they may be formulated as injections (intramuscular, subcutaneous, intramedullary, intrathecal, intraventricular, intraperitoneal, intravenous), drop infusion preparations, or suppositories. For application by the ophthalmic mucous membrane route, they may be formulated as eye drops or eye ointments. These formulations can be prepared by conventional means, and, if desired, the active compound (i.e., adenosine receptor antagonists) may be mixed with any conventional additive, such as an excipient, a binder, a disintegrating agent, a lubricant, a corrigent, a solubilizing agent, a suspension aid, an emulsifying agent, or a coating agent.

### Adenosine Receptor Antagonists

Suitable adenosine receptor antagonists can be, for example, ZM241385 (4-(2-(7-amino-2-(furan-2-yl)-[1,2,4]triazolo[1,5-a][1,3,5]triazin-5-ylamino)ethyl)phenol), ATL-444 ((1S,3R)-1-[2-(6-amino-9-prop-2-ynylpurin-2-yl)ethynyl]-3-methylcyclohexan-1-ol), istradefylline (KW-6002; 8-[(E)-2-(3,4-dimethoxyphenyl)vinyl]-1,3-diethyl-7-methyl-3,7-dihydro-1H-purine-2,6-dione), MSX-3, preladenant (SCH-420,814), SCH-58261 (5-Amino-7-(2-phenylethyl)-2-(2-furyl)-pyrazolo(4,3-e)-1,2,4-triazolo(1,5-c)pyrimidine), SCH-412,348, SCH-442,416 (2-(2-furyl)-7-[3-(4-methoxyphenyl)propyl]-7H-pyrazolo[4,3-e][1,2,4]triazolo[1,5-c]pyrimidin-5-amine), ST-1535, caffeine, VER-6623, VER-6947, VER-7835, vipadenant (BIIB-014), theophylline (1,3-dimethyl-7H-purine-2,6-dione), ATL-801, 1,3-Dialkyl-8-(hetero)aryl-9-OH-9-deazaxanthine (compound 38), CVT-6883, MRS-1706 (N-(4-Acetylphenyl)-2-[4-(2,3,6,7-tetrahydro-2,6-dioxo-1,3-dipropyl-1H-purin-8-yl)phenoxy]acetamide), MRS-1754, OSIP-339,391, PSB-603, PSB-0788, PSB-1115 and combinations thereof. It should be understood in the art, that the adenosine receptor antagonists may be administered in any suitable form known in the art to achieve the desired therapeutic effect, such as, for example, free acid, free base, and salts thereof. In one particularly suitable embodiment, the adenosine receptor antagonist is ZM241385, commercially available from Tocris Bioscience, United Kingdom, which specifically binds to both adenosine 2a receptors and adenosine 2b receptors.

Actual dosage levels of the adenosine receptor antagonist in a pharmaceutical formulation for use in the methods of the present disclosure may be varied so as to obtain an amount of the adenosine receptor antagonist that is effective to achieve the desired therapeutic response or benefit for a particular subject, formulation, and/or mode of administration. More particularly, as used herein, the phrase “therapeutically effective amount” of the adenosine receptor antagonist used in the methods of the present disclosure refers to a sufficient amount of the adenosine receptor antagonist to treat bleeding disorders as defined herein, at a reasonable benefit/risk ratio applicable to any medical treatment. It can be understood, however, that the total daily usage of the adenosine receptor antagonist and pharmaceutical formulations including the adenosine receptor antagonists for use in the methods of the present disclosure can be decided by the attending physician within the scope of sound medical judgment. The specific therapeutically effective dose level for any particular subject can depend upon a variety of factors including the bleeding disorder being treated and the severity of the bleeding disorder; activity of the specific adenosine receptor antagonist employed; the specific pharmaceutical formulation employed; the age, body weight, general health, sex and diet of the subject; the time of administration, route of administration, and rate of excretion of the specific adenosine receptor antagonist employed; the duration of the treatment; drugs used in combination or coincidental with the specific adenosine receptor antagonist employed; and like factors well-known in the medical arts. For example, it is well within the skill of the art to start doses of the adenosine receptor antagonist at levels lower than required to achieve the desired therapeutic effect and to gradually increase the dosage until the desired effect is achieved.

In some embodiments, the adenosine receptor antagonist can be administered intraperitoneally to a subject in need thereof in an amount ranging from about 100 mg/kg total body weight of the subject to about 300 mg/kg total body weight of the subject per day. In one embodiment, the methods of the present disclosure include intravenously administering to a subject in need thereof an amount of about 100 mg/kg total body weight of the subject per day. In some embodiments, the adenosine receptor can be administered to a subject in need thereof in daily amounts of from about 1 mg to about 34 g, including from about 1 mg to about 20 g, and including from about 1 mg to about 16 g. In one particular embodiment, the adenosine receptor can be administered to a subject in need thereof orally in daily amounts of from about 1 mg to about 16 g. The daily dosage of adenosine receptor antagonists or pharmaceutical formulation including the adenosine receptor antagonists may be in the form of a single dosage or may be in the form of two dosages, three dosages, four dosages or more to be administered two or more times during the day.

### Factors VIII, IX and XI

In some embodiments, the methods of the present disclosure include co-administering the adenosine receptor antagonists described above with a therapeutically effective amount of one or more of Factor VIII, Factor IX and Factor XI. Factor VIII, also known as anti-hemophilic factor (AHF), is a glycoprotein pro-cofactor produced in liver sinusoidal cells and endothelial cells outside of the liver throughout the body. This protein circulates in the bloodstream in an inactive form, bound to von Willebrand factor, until an injury that damages blood vessels occurs. In response to an injury, Factor VIII is activated and dissociates from von Willebrand factor. The active protein (sometimes written as Factor VIIIa) interacts with Factor IX in the coagulation cascade to activate Factor X. As noted above, Factor X then mediates the generation of thrombin from prothrombin, with the ultimate production of fibrin from fibrinogen to allow for clot formation.

Factor IX is a zymogen (proenzyme) that is processed to remove the signal peptide, glycosylated and then cleaved by Factor IXa of the contact pathway or Factor VIIa of the tissue factor pathway to produce a two-chain form where the chains are linked by a disulfide bridge. When activated into Factor IXa, in the presence of Ca²⁺, membrane phospholipids, and a Factor VIII cofactor, it hydrolyses one arginine-isoleucine bond in Factor X to form Factor Xa, which mediates the generation of thrombin from prothrombin as noted above. More particularly, Factor IX is a serine protease composed of four protein domains, a Gla domain, two tandem copies of the EGF domain, and a C-terminal trypsin-like peptidase domain, which carries out the catalytic cleavage. The N-terminal EGF domain has been shown to at least in part be responsible for binding tissue factor. Further, residues 88 to 109 of the second EGF domain mediate binding to platelets and assembly of the Factor X activating complex.

Factor XI (plasma thromboplastin antecedent) is the zymogen (proenzyme) form of Factor XIa. Factor XI circulates as a homodimer in an inactive form. Factor XI is activated into Factor XIa via cleavage by Factor XIIa, thrombin and autocatalysis. Active Factor XIa is a serine protease that activates Factor X to mediate the generation of thrombin from prothrombin as noted above.

In one embodiment, the methods include co-administering the antagonist with recombinant Factor VIII, recombinant Factor IX, recombinant Factor XI and combinations thereof. Recombinant Factor VIII, recombinant Factor IX, and recombinant Factor XI refers to their production by protein expression methods and chemical synthesis methods. Protein expression methods for producing recombinant proteins are known to those skilled in the art. Generally, “recombinant Factor VIII”, “recombinant Factor IX” and “recombinant Factor XI” used herein to describe Factor VIII, Factor IX, and Factor XI polypeptides, which by virtue of their origin or manipulation, may not be associated with all or a portion of the polypeptide with which they are associated in nature and/or is linked to a polypeptide other than that to which they are linked in nature. Recombinant Factor VIII, recombinant Factor IX and recombinant Factor XI may not necessarily be translated from a designated nucleic acid sequence. For example, the recombinant Factor VIII, recombinant Factor IX and recombinant Factor XI can also be generated in any manner such as, for example, chemical synthesis methods including, for example, liquid-phase peptide synthesis, solid-phase peptide synthesis, fragment condensation, and chemical ligation.

One particularly suitable Factor VIII is commercially available as Kogenate FS (Bayer HealthCare Pharmaceuticals, Germany). Particularly suitable Factor IXsources are commercially available as Benefix® (Pfizer, New York, N.Y.) and Mononine® (CSL Behring, Sweden).

When co-administered with the adenosine receptor antagonist, Factor VIII, Factor IX and/or Factor XI can be administered in varying amounts such to be effective to achieve the desired therapeutic response for a particular subject, pharmaceutical formulation, and/or mode of administration. As used herein, the phrase “therapeutically effective amount” of Factor VIII, Factor IX and/or Factor XI used in the methods of the present disclosure refers to a sufficient amount of Factor VIII, Factor IX and/or Factor XI to be used in combination with the adenosine receptor antagonist to improve the performance of the adenosine receptor antagonist in treating bleeding disorders as defined herein, at a reasonable benefit/risk ratio applicable to any medical treatment. It can be understood, however, that the total daily usage of Factor VIII, Factor IX and/or Factor XI and pharmaceutical formulations including adenosine receptor antagonist and Factor VIII, Factor IX and/or Factor XI for use in the methods of the present disclosure can be decided by the attending physician within the scope of sound medical judgment. The specific therapeutically effective dose level for any particular subject may depend upon a variety of factors including the bleeding disorder being treated and the severity of the bleeding disorder; the specific pharmaceutical formulation employed; the age, body weight, general health, sex and diet of the subject; the time of administration, and route of administration; the duration of the treatment; specific adenosine receptor antagonist used in combination with Factor VIII, Factor IX and/or Factor XI; and like factors well-known in the medical arts. For example, it is well within the skill of the art to start doses of Factor VIII, Factor IX and/or Factor XI at levels lower than required to achieve the desired therapeutic effect and to gradually increase the dosage until the desired effect is achieved.

In some embodiments, Factor VIII can be co-administered with the adenosine receptor antagonist in an amount ranging from about 0.5 IU/kg total body weight of the subject to about 50 IU/kg total body weight of the subject per day.

In some embodiments, Factor IX can be co-administered with the adenosine receptor antagonist in an amount ranging from about 0.5 IU/kg total body weight of the subject to about 50 IU/kg total body weight of the subject per day.

The daily dosage of Factor VIII, Factor IX and/or Factor XI to be co-administered with the adenosine receptor antagonist may be in the form of a single dosage or may be in the form of two dosages, three dosages, four dosages or more to be administered two or more times during the day.

Advantageously, the methods of the present disclosure allow for restoring hemostasis for bleeding indications by inhibiting targets unrelated to the coagulation cascade.

The disclosure will be more fully understood upon consideration of the following non-limiting Example.

### Example

In this Example, the effect of administering an adenosine receptor antagonist on reduction in blood loss in mice with Hemophilia A and Hemophilia B mice was analyzed.

Male Hemophilia A or Hemophilia B mice, approximately eight to nine weeks of age and weighing, on average, approximately 25 grams, were used in this Example. The mice were anesthetized with isoflurane and the tails were placed in approximately 37-38° C. warmed 15-ml plastic tubes including 0.9% (by weight) saline for 10 minutes. The tails were then cut at 4 mm from the tip by scalpel and immediately placed back into separate pre-warmed (37-38° C.) 15-ml plastic tubes containing 10 ml of 0.9% (by weight) saline. Each mouse was allowed to bleed freely for 30 minutes.

The adenosine 2a and 2b receptor antagonist, 4-(2-[7-Amino-2-(2-furyl)[1,2,4]triazolo[2,3-a][1,3,5]triazin-5-ylamino]ethyl)phenol (commercially available as ZM241385 from Tocris Bioscience, United Kingdom), was used in this Example. In a first sample group, ZM241385 was dissolved in 100% DMSO. In a second sample group, ZM241385 was dissolved in PEG400/ethanol/water (40/10/50 v/v/v). In a third sample group, ZM241385 was dissolved in PEG400/water (60/40 v/v). Mice having Hemophilia A were injected (10 mice/sample antagonist solution) with either: the first sample adenosine receptor antagonist solution by intraperitoneal injection in the amount of 100 mg/kg ZM241385 thirty minutes prior to the tail cut, or the second sample adenosine receptor antagonist solution by intraperitoneal injection in the amount of 300 mg/kg ZM241385 thirty minutes prior to the tail cut. Control mice were treated (10 mice/control treatment) with either: 100 μl 100% DMSO or 300 μl of PEG400/ethanol/water (40/10/50 v/v/v) without any adenosine receptor antagonist prior to the tail cut to serve as untreated controls.

Nineteen mice having Hemophilia B were injected with the third sample of adenosine receptor antagonist solution by intravenous injection in the amount of 100 mg/kg ZM241385 five minutes prior to the tail cut. Nineteen control mice were treated with 100 μl of PEG400/water (60/40 v/v) without any adenosine receptor antagonist prior to the tail cut to serve as an untreated control.

Blood loss was quantified gravimetrically by weighing the tubes before and after blood was collected. The results are shown in FIGS. 2-4.

As shown in FIGS. 2-4, inhibition of adenosine receptors by ZM241385 reduced blood loss in both mice having Hemophilia A and mice having Hemophilia B. These results demonstrate that inhibition of targets unrelated to the coagulation cascade, such as adenosine receptors, can provide for pro-hemostatic effects.

1. A method for treating a bleeding disorder in a subject in need thereof, the method comprising administering a therapeutically effective amount of an adenosine receptor antagonist.

2. The method of claim 1 wherein the adenosine receptor is selected from the group consisting of an adenosine 2a receptor, an adenosine 2b receptor and combinations thereof.

3. The method of claim 1 wherein the adenosine receptor antagonist is selected from the group consisting of ZM241385 (4-(2-(7-amino-2-(furan-2-yl)-[1,2,4]triazolo[1,5-a][1,3,5]triazin-5-ylamino)ethyl)phenol), ATL-444 ((1S,3R)-1-[2-(6-amino-9-prop-2-ynylpurin-2-yl)ethynyl]-3-methylcyclohexan-1-ol), istradefylline (KW-6002; 8-[(E)-2-(3,4-dimethoxyphenyl)vinyl]-1,3-diethyl-7-methyl-3,7-dihydro-1H-purine-2,6-dione), MSX-3, preladenant (SCH-420,814), SCH-58261 (5-Amino-7-(2-phenylethyl)-2-(2-furyl)-pyrazolo(4,3-e)-1,2,4-triazolo(1,5-c)pyrimidine), SCH-412,348, SCH-442,416 (2-(2-furyl)-7-[3-(4-methoxyphenyl)propyl]-7H-pyrazolo[4,3-e][1,2,4]triazolo[1,5-c]pyrimidin-5-amine), ST-1535, caffeine, VER-6623, VER-6947, VER-7835, vipadenant (BIIB-014), theophylline (1,3-dimethyl-7H-purine-2,6-dione), ATL-801, 1,3-Dialkyl-8-(hetero)aryl-9-OH-9-deazaxanthine (compound 38), CVT-6883, MRS-1706 (N-(4-Acetylphenyl)-2-[4-(2,3,6,7-tetrahydro-2,6-dioxo-1,3-dipropyl-1H-purin-8-yl)phenoxy]acetamide), MRS-1754, OSIP-339,391, PSB-603, PSB-0788, PSB-1115 and combinations thereof.

4. The method of claim 1 further comprising co-administering a therapeutically effective amount of an agent selected from the group consisting of Factor VIII, Factor IX, and combinations thereof.

5. The method of claim 4 wherein the Factor VIII and Factor IX are recombinant Factor VIII and recombinant Factor IX.

6. The method of claim 1 wherein the bleeding indication is selected from the group consisting of Hemophilia A, Hemophilia B, Factor VIII deficiency, Factor XI deficiency, von Willebrand Disease, Glanzmann's Thrombasthenia, Bernard Soulier Syndrome, idiopathic thrombocytopenic purpura, and trauma.

7. The method of claim 1 wherein the adenosine receptor antagonist is administered to the subject in need thereof in an amount of from about 1 mg to about 16 g per day.

8. The method of claim 1 wherein the treating includes reducing bleeding time in the subject in need thereof.

9.-18. (canceled)

19. A method for enhancing platelet aggregation in response to adenosine diphosphate (ADP) in a subject in need thereof, the method comprising administering a therapeutically effective amount of an adenosine receptor antagonist.

20. The method of claim 19 wherein the adenosine receptor is selected from the group consisting of an adenosine 2a receptor, an adenosine 2b receptor and combinations thereof.

21. The method of claim 19 wherein the adenosine receptor antagonist is selected from the group consisting of ZM241385 (4-(2-(7-amino-2-(furan-2-yl)-[1,2,4]triazolo[1,5-a][1,3,5]triazin-5-ylamino)ethyl)phenol), ATL-444 ((1S,3R)-1-[2-(6-amino-9-prop-2-ynylpurin-2-yl)ethynyl]-3-methylcyclohexan-1-ol), istradefylline (KW-6002; 8-[(E)-2-(3,4-dimethoxyphenyl)vinyl]-1,3-diethyl-7-methyl-3,7-dihydro-1H-purine-2,6-dione), MSX-3, preladenant (SCH-420,814), SCH-58261 (5-Amino-7-(2-phenylethyl)-2-(2-furyl)-pyrazolo(4,3-e)-1,2,4-triazolo(1,5-c)pyrimidine), SCH-412,348, SCH-442,416 (2-(2-furyl)-7-[3-(4-methoxyphenyl)propyl]-7H-pyrazolo[4,3-e][1,2,4]triazolo[1,5-c]pyrimidin-5-amine), ST-1535, caffeine, VER-6623, VER-6947, VER-7835, vipadenant (BIIB-014), theophylline (1,3-dimethyl-7H-purine-2,6-dione), ATL-801, 1,3-Dialkyl-8-(hetero)aryl-9-OH-9-deazaxanthine (compound 38), CVT-6883, MRS-1706 (N-(4-Acetylphenyl)-2-[4-(2,3,6,7-tetrahydro-2,6-dioxo-1,3-dipropyl-1H-purin-8-yl)phenoxy]acetamide), MRS-1754, OSIP-339,391, PSB-603, PSB-0788, PSB-1115 and combinations thereof.

22. The method of claim 19 further comprising co-administering a therapeutically effective amount of an agent selected from the group consisting of Factor VIII, Factor IX, and combinations thereof.

23. The method of claim 22 wherein the Factor VIII and Factor IX are recombinant Factor VIII and recombinant Factor IX.

24.-28. (canceled)

29. A method for increasing platelet surface coagulation in a subject in need thereof, the method comprising administering a therapeutically effective amount of an adenosine receptor antagonist.

30. The method of claim 29 wherein the adenosine receptor is selected from the group consisting of an adenosine 2a receptor, an adenosine 2b receptor and combinations thereof.

31. The method of claim 29 wherein the adenosine receptor antagonist is selected from the group consisting of ZM241385 (4-(2-(7-amino-2-(furan-2-yl)-[1,2,4]triazolo[1,5-a][1,3,5]triazin-5-ylamino)ethyl)phenol), ATL-444 ((1S,3R)-1-[2-(6-amino-9-prop-2-ynylpurin-2-yl)ethynyl]-3-methylcyclohexan-1-ol), istradefylline (KW-6002; 8-[(E)-2-(3,4-dimethoxyphenyl)vinyl]-1,3-diethyl-7-methyl-3,7-dihydro-1H-purine-2,6-dione), MSX-3, preladenant (SCH-420,814), SCH-58261 (5-Amino-7-(2-phenylethyl)-2-(2-furyl)-pyrazolo(4,3-e)-1,2,4-triazolo(1,5-c)pyrimidine), SCH-412,348, SCH-442,416 (2-(2-furyl)-7-[3-(4-methoxyphenyl)propyl]-7H-pyrazolo[4,3-e][1,2,4]triazolo[1,5-c]pyrimidin-5-amine), ST-1535, caffeine, VER-6623, VER-6947, VER-7835, vipadenant (BIIB-014), theophylline (1,3-dimethyl-7H-purine-2,6-dione), ATL-801, 1,3-Dialkyl-8-(hetero)aryl-9-OH-9-deazaxanthine (compound 38), CVT-6883, MRS-1706 (N-(4-Acetylphenyl)-2-[4-(2,3,6,7-tetrahydro-2,6-dioxo-1,3-dipropyl-1H-purin-8-yl)phenoxy]acetamide), MRS-1754, OSIP-339,391, PSB-603, PSB-0788, PSB-1115 and combinations thereof.

32. The method of claim 29 further comprising co-administering a therapeutically effective amount of an agent selected from the group consisting of Factor VIII, Factor IX, and combinations thereof.

33. The method of claim 32 wherein the Factor VIII and Factor IX are recombinant Factor VIII and recombinant Factor IX.

34. The method of claim 19 wherein the adenosine receptor antagonist is administered to the subject in need thereof in an amount of from about 1 mg to about 16 g per day.

35. The method of claim 29 wherein the adenosine receptor antagonist is administered to the subject in need thereof in an amount of from about 1 mg to about 16 g per day.
